(19) United States
(12) Patent Application Publication
(10) Pub. No.: US 2008/0044916 A1
(43) Pub. Date: Feb. 21, 2008

(54) Computational selection of probes for localizing chromosome breakpoints

(76) Inventors: Peter K. Rogan, Overland Park, KS (US); Joan Knoll, Overland Park, KS (US)

Correspondence Address: ERICKSON & KLEYPAS, L.L.C., 800 W. 47TH STREET, SUITE 401, KANSAS CITY, MO, 64112, US

(21) Appl. No.: 11/091,860

(22) Filed: Mar. 28, 2005

Related U.S. Application Data
(60) Provisional application No. 60/557,007, filed on Mar. 26, 2004.

(51) Int. Cl. G01N 33/00; C07H 21/04

(52) U.S. Cl. 436/94; 536/24.3

(57) ABSTRACT

The present invention provides a novel method for selecting genomic hybridization probes. The method generally includes the step of selecting a genomic interval of interest, identifying at least one potential hybridization probe that has known coordinates within the interval of interest, applying a numerical method to sample the identified probe(s) based on its genome coordinates, and selecting a probe based on the result of the numerical method.

### RELATED APPLICATIONS

This application claims the benefit of provisional application Ser. No. 60/557,007 filed on Mar. 26, 2004, the teachings and content of which are incorporated by reference herein. Additionally, the teachings and content of Document Disclosure No. ______, filed on Jan. 17, 2004, are specifically incorporated by reference herein.

### GOVERNMENT INTEREST

This invention was made with Government support under grant CA 095167 from the National Institutes of Health. The United States Government may have certain rights specifically with regard to the single copy probes described in the claimed invention.

### BACKGROUND OF THE INVENTION

One common cause of cancer and other genetic disorders in plants and animals is the rearrangement of chromosomes. For example, a genetic sequence usually found in one chromosome is instead translocated to a different chromosome. Despite knowledge of the role these rearrangements play, no previously available methods have been suitable for the throughput and scale of a large scale project such as the Human Cancer Genome Project. (White Paper, Recommendation for a Human Cancer Genome Project, Report of Working Group on Biomedical Technology, U.S. National Cancer Institute Advisory Board, February 2005, the teachings and content of which are hereby incorporated by reference herein). In that White Paper, it was admitted that currently available methods for detecting chromosomal rearrangements suffer from a multitude of problems. For example, multi-color fluorescent hybridization requires whole cell preparations, not just genomic DNA and has relatively low resolution, while shotgun sequencing of paired ends from large DNA fragments is too costly for large-scale application.

In addition to the problems noted above, detection of such chromosomal rearrangements has traditionally required the identification of recombinant DNA clones that contain the rearrangement. Such clones are derived from genetic libraries created from a patient's DNA. This procedure involves identification of clones containing sequences on each side of the break. In the case of cancer, this usually involves selection of clone from two different genes that have fused. Typically, probes for this procedure are selected based on restriction sites; the order of the sites is either mapped within a clone or based on sequences specifically known to contain one or more exons that were previously identified by hybridization to a cDNA segment. This enables identification of genomic clones containing a breakpoint, but will not localize the breakpoint within the clone. To achieve higher resolution, it is necessary to use sub-sequences from the probes to determine the location of the breakage interval within the clone. In molecular genetic approaches, breakpoints are identified by length variation in restriction products using Southern analysis or by examination of PCR products of abnormal size or composition. The selection of probes is not generally based on genome coordinates, but is rather based on local coordinates within the clone or other distinguishing features within the clone, gene, or GenBank accession number, which serve as landmarks for sequence mapping.

Recombinant genomic clones or even fragments derived from such clones spanning a chromosome breakpoint can detect the presence of a chromosome rearrangement (for example, see U.S. Pat. No. 6,576,421 and U.S. Pat. No. 6,344,315, the teachings and content of which are hereby incorporated by reference herein). However, these methods do not provide adequate resolving power to delineate the location of the breakpoint interval at high resolution. Additionally, the prior art does not teach the determination of genomic coordinates of the breakpoint interval, because the sequences of such genomic clones were not mapped precisely onto the genome reference sequence. Other prior art methods use genomic probes that may directly hybridize to chromosomes in order to detect rearrangements, but these methods do not map such probes onto genome reference coordinates, nor do they determine the intervals containing the juxtaposed genomic sequences.

Another prior art method of detecting breakpoints involves using cDNA to localize breakpoints or breakpoint intervals within a gene to a particular exon, but not to the genome itself. This involves determining the genomic locations of sequence defined sub-segments based on their sequence homology to the cDNA segment by either fluorescent in situ hybridization (FISH), Southern or array comparative genomic hybridization. This provides an indirect assignment of the approximate location of the breakpoint, since the abnormal pattern of hybridization to the chromosomal sequences will delineate which exons in the mRNA that is derived from the chromosome are found in their normal context. It does not reveal the genomic coordinates or distances between genomic segments that hybridize to different portions of the cDNA. However, this method does not determine the coordinates of the breakpoint in the chromosome. This is because adjacent exons may be widely separated by large introns in eukaryotic genomes (see U.S. Pat. No. 6,040,140 to Croce et al., the teachings and content of which are hereby incorporated by reference herein). Thus, the range of coordinates defining the breakage interval may be so large that it has little utility in defining breakpoints. For example, it is not uncommon in the human and murine genomes for entire genes to be nested within the introns of other genes. The failure to delineate the genomic interval of breakage at high resolution using cDNA sub-sequences as probes introduces ambiguity into the determination as to whether the original gene or its nested counterpart is disrupted by a chromosome rearrangement.

Another prior art method for breakpoint delineation involves amplification-based approaches such as vectorette and pan-handle polymerase chain reaction (for example, see U.S. Pat. No. 6,368,791, the teachings and content of which are hereby incorporated by reference herein) to identify sequences at the junctions of chromosome rearrangements. These methods enable the retrieval of a previously unknown genomic sequence adjacent to a known sequence. However, they require that the known sequence occur within several kilobase pairs of the unknown sequence. Thus, these methods do not provide a means of defining a breakpoint using multiple probes over longer genomic distances. The site of chromosomal rearrangements can be inferred if the known sequence is juxtaposed with an unknown sequence that is derived from a different chromosome or a novel location on the same chromosome. The position of the junction is inferred by comparison of the sequence carrying the rearrangement with the corresponding sequence from the normal reference genome. These procedures do not require prior knowledge of the coordinates of the genomic segment.

Accordingly, methods for detecting the precise genomic location of breakpoints within a chromosome are needed in the art. Moreover, methods for efficiently selecting probes to be used in such methods are also needed in order to minimize the number of probes used to define a breakpoint location or interval.

### SUMMARY OF THE INVENTION

The present invention solves the problems inherent in the prior art and provides a distinct advance in the state of the art by providing methods for localization of genomic intervals containing the boundaries of genomic rearrangements. By delineating these boundaries, it is possible to determine precisely the nature of a chromosome abnormality in a patient with an inherited or acquired genomic disorder, to identify the boundaries of polymorphic segments in normal individuals that differ in copy number, or to reveal the genetic basis for pathogenic or nonpathogenic traits in plants (see, B. McClintock, The Origin and Behavior of Mutatable Loci in Maize, 36(6) PNAS, 344-355 (1950) and E. D. Badaeva, Molecular Cytogenetic Analysis of Tetraploid and Hexaploid Aegilops Crassa, 6(8) Chromosome Res., 629-637 (1998), the teachings and content of which are hereby incorporated by reference) or animals See, C. Herens, Cytogenetic Changes in Hepatocarcinomas from Rats Treated with Chronic Exposure to Diethylnitrosamine, 60(1) Cancer Genet Cytogenet., 45-52, (1992), especially in inbred laboratory strains or breeding stock (see M. Liyanage et al., Multicolour Spectral Karotyping of Mouse Chromosomes, 14(3) Nat. Genet., 312-315 (1996), the teachings and content of which are hereby incorporated by reference). The nature of the abnormality can reveal missing or extra copies of individual or multiple genes, defined as partial aneuploidy found in unbalanced chromosome rearrangements or the intervals bracketing balanced chromosome rearrangements, typically found in translocations or inversions that disrupt one or more genes within the breakpoint intervals. In some instances, the rearrangements can even disrupt the regulatory sequences that control the expression or developmental program of genes (rather than the gene itself), thereby disrupting the timing or tissue specificity of gene expression.

The present invention exploits the coordinates of genomic probes in genome reference sequences to provide methods of selecting probes for delineation of those intervals that are adjacent to a chromosome breakpoint. It is not necessary to have a complete genome reference sequence in order to practice this invention, only a complete sequence of a particular region within which the boundary of the rearrangement resides. The invention selects probes that bracket this particular chromosomal interval containing a breakpoint by first identifying a series of potential probes in the genomic region containing the breakpoint. This region may span millions of nucleotides in length, for example, covering a chromosomal band in the human genome, and therefore numerous probes may be used to define the location of the break. Next, the instant invention provides a means of selecting amongst the universe of potential probes in order to efficiently and quickly narrow the breakpoint to a small interval. Even if the specific breakpoint sequence is not ultimately determined, by limiting the breakpoint interval to a region of up to approximately 25 kilobase pairs (see Example 1), it is often feasible to determine from available genome annotation precisely which gene or genes has been disrupted and the approximate location within the gene where the break has occurred. This information can be valuable in predicting either the clinical phenotype of a patient or the degree to which the gene function may be impaired in an animal or plant model or strain. In some preferred forms of the present invention, the interval of interest or target region is associated with a known chromosomal breakpoint.

In carrying out the methods of the present invention, computational procedures for localizing the intervals or sites containing chromosomal breaks are used. These procedures will assist in minimizing the number of assays required to define small genomic intervals containing chromosomal breakpoints or rearrangements. Another advantage of the present invention lies in the use of probes that are precisely defined in terms of location within the human genome reference sequence. Once the interval within which a break occurs has been localized, the sequences of the breakpoints can be determined to single nucleotide resolution by conventional molecular biological methods such as panhandle PCR and DNA sequencing.

In general, the present invention provides a method of selecting a genomic hybridization probe with the method generally including the steps of selecting a genomic interval of interest, identifying a plurality of potential hybridization probes of known genomic coordinates in the interval of interest, applying a numerical method to sample the plurality of probes based on their genome coordinates, and selecting a probe based on the results of the numerical sampling method. The genomic hybridization probe can be a “single copy” genomic probe wherein “single copy” refers to a sequence which is strictly unique (i.e., which is complementary to one and one only sequence in the corresponding genome) but also covers duplicons and triplicons. Stated otherwise, a “single copy” probe in preferred forms will hybridize to three or less locations in the genome. Preferably, the single copy probes of the invention should have a length of at least about 50 nucleotides, and more preferably at least about 100 nucleotides. Probes of this length are sufficient for Southern blot analyses, bead array suspension hybridization, microarray hybridization, multiplex amplifiable probe hybridization and other hybridization techniques. However, if other analyses such as FISH are employed, the probes should be somewhat longer, i.e., at least about 500 nucleotides, still more preferably at least about 1000 nucleotides in length, even more preferably at least about 1500 nucleotides in length, and still even more preferably at least about 2000 nucleotides in length. Single copy probes are typical of the probes suitable for use with the present invention due to their broad and very dense genomic distribution and well defined unique genomic coordinates. Those of skill in the art will appreciate that smaller probes can provide greater resolution of the precise breakpoint location, provided there is sufficient density of probes within a region of interest or target region. It will also be appreciated that non-single copy probes also find great utility in the present invention, provided that their boundary coordinates, i.e., their endpoint coordinate on each side, are known and defined to specific coordinates in the genome. These non-single copy probes can contain interspersed repetitive sequences as well as single copy stretches of nucleic acids. Such probes may need to have blocking or masking nucleic acids (such as Cₒt−1 DNA) preannealed thereto and used prior to chromosomal or genomic hybridization in order to prevent or reduce cross hybridization of repetitive sequences to other locations in the genome. Moreover, if non-single copy probes are used, it is preferred to also use at least some single-copy probes, because if non single copy probes composed entirely of repetitive sequences are used, the delineation or localization of rearrangements or breakpoints will be difficult due to the inability to assign the hybridization to any particular set of genomic coordinates. For purposes of the present invention, it is also preferable to select probes that do not contain overlapping chromosome coordinates. If probes with overlapping chromosome coordinates are used, it is possible that any results coming therefrom could be ambiguous.

The ability of the present invention to precisely define a genomic or chromosomal interval containing a breakpoint or rearrangement is dependent upon the density of probes within the region of interest or target region surveyed. Higher resolution and precision are afforded by a sufficiently high density of probes within the region. Prior art recombinant genomic probes, especially those available commercially, are considerably larger (generally between 50 and 600 kilobase pairs) than those typically used with the instant invention. The single copy probes and single copy with interspersed repeat probes of the art are suitable for this method because they are present at adequate densities to precisely localize a chromosomal breakpoint such that the breakpoint itself can be efficiently and quickly determined subsequently by genomic restriction digestion, amplification techniques such as panhandle or vectorette PCR, and dideoxy sequencing of the fragments containing the DNA junction linking two sequences that are ordinarily not colinear on the chromosome.

Probes are selected within an interval based on numerical methods including mathematical formulae, algorithms, sampling methods, neural network approaches including heuristic Markov models, Gibbs sampling, greedy algorithms, supervised and non-supervised learning methods, information theory based models of protein binding sites within genomic sequences, and the like, that determine the location of the probe to choose. Despite the considerably higher resolution of the instant method, it is not always feasible to select a probe precisely at the coordinate computed by the numerical method because the preferred set of coordinates occurs within a long stretch of highly reiterated sequences. In such instances, the closest or nearest single copy probe or combination of multiple single copy and interspersed repeat probes is instead selected. The results of applying those numerical methods produce a result, ie. whether the probe signal remains on the original portion of the chromosome (as defined by the presence of the original centromere) or if it is missing from that normal context due to, for example, either deletion, translocation to another chromosome, or it appears at a different location on the same chromosome due to translocation or inversion. Another possibility is that there is local amplification of the sequence in the same chromosomal domain or additional copies amplified in a new chromosomal context. If the probe detects the chromosomal sequence on the original portion of the chromosome, ie. the derivative chromosome, the probe is said to stay (st) on this chromosome (this is an expected result). If it occurs elsewhere on the chromosome or on other chromosomes, it is said to move (mv). If a copy of the sequence is missing from the genome, the probe is said to be deleted (del) or if the break occurs with a probe or set of probes, then the probe(s) are said to split (spl) or separate (sep). Additional copies of the probe are indicated with the number of copies detected (eg. ×2 for two copies).

As each probe (or probe combination) is tested, preferred methods of the present invention score probes for the expected (st) versus the other possible outcomes (mv, del, spl, add). The breakpoint is delineated by probes that are collinear in a normal chromosome but have discordant outcomes when hybridized to chromosomes from a patient that harbors a chromosome rearrangement. The objective of the method is to hybridize a series or combination of probes that are colinear in normal individuals and to progressively, iteratively apply closer to one another on normal chromosomes so as to delineate the smallest possible pair of intervals with discordant scoring patterns.

A plurality of numerical or mathematical methods can be used to select probes to localize chromosomal breaks. All of these strategies have in common the requirement to identify one or more probes with discordant chromosomal scoring patterns. The instant invention teaches that certain numerical approaches may be more efficient, depending the number of breakpoint intervals that have been previously ascertained. The selection of probes based on prior probability of a breakpoint occurring within a particular interval requires that there have already been observations of breaks within that interval. In the absence of such information, it is more appropriate to apply numerical methods that select probes based on genome coordinates alone which progressively reduce the size of the interval bounded by probes with discordant chromosomal scoring patterns. Examples are provided of bisection and golden ratio section methods which dictate the locations of probes based on well established numerical formulae. It is important to note that none of the methods of the instant invention saturate the testing of the chromosomal interval with all of the available probes, as this would be an extremely inefficient approach to delineating the chromosomal breakpoint interval.

The present invention is also advantageous because it is based on the coordinates of known genomic sequences. Therefore, it is feasible to determine the maximum and possible minimum size of the genomic interval with greater precision than is feasible with commercially available or other recombinant probes typically used for chromosomal hybridization. Another advantage is that numerical methods based on the chromosomal coordinates themselves for bracketing the breakpoint interval can be applied to determine the order of probes used to more narrowly delineate the interval. Linear minimization methods for determining shortest paths are well known in computer science and other applied mathematical applications, but heretofore have not been anticipated or applied towards chromosomal localization of breakpoints, despite the admitted problems inherent with the prior art approaches.

In preferred forms, once the probe or probes have been selected based on the numerical methods, the general method further includes the step of hybridizing the selected probe with the selected interval.

In practice, the numerical method selected for use with the present invention can be any method that helps to select a probe for use in delineating the interval within which a breakpoint or rearrangement occurs, and thereby avoids the “brute-force” approach of using every available probe within an interval to delineate the interval containing the breakpoint or rearrangement. Some preferred methods include the general bisection method, dichotomous (divides into equal parts) bisection method, golden section ratio method, combinatorial bracketing, cumulative probability, and combinations thereof.

One preferred numerical method is referred to as the bisection method of probe selection. In the bisection method, an interval is selected and is divided into two nearly-even sections. A probe near the bisection coordinate is selected for hybridization and then, in cytogentic applications of balanced chromosome rearrangements, it is determined whether the probe moves or stays. The results of this hybridization will determine which interval is next selected for a second bisection. In the situation involving deletions or amplifications (partial aneuploidies), only probes that are present that are not deleted can be scored and the abnormality is detected by determining how many hybridization signals are present. For context independent hybridization methods, such as microarrays, bead array suspension, Southern blotting, and multiplex amplifiable probe hybridization, the ratio of intensities for deleted versus non-deleted loci is used to score the results of the experiment. However, the computational method of selecting the probes for these context independent methods is indistinguishable from the techniques used for cytogenetic (e.g. FISH) probe selection.

A variation of the bisection method involves a two point (dichotomous, meaning to divide into 2 parts) search for finding a solution to f(x) in order to determine the location of a chromosome breakpoint. f(x) is a Boolean function. It equals 0 if there is no break detected by the probe—that is, if the probe stays with the original derivative chromosome. It equals 1 if there is a break detected by the probe—that is, if the probe moves to the second derivative chromosome or the probe signal is split or separated (which would indicate that the breakpoint is located in the sequence hybridized by the probe).

In order to find a solution to minimize f(x), first one selects a chromosomal interval with known endpoints a and b. The first probe selected will be x1, which has a first endpoint that is approximately equal to a+(b−a)/2−ε/2, where E is the resolution of the probes. The second probe selected will be x2, which should have a first endpoint that is approximately equal to a+(b−a)/2+ε/2. The two probes are then hybridized to the chromosomal interval and the results examined and plugged into the Boolean function. Then, the solutions to f(x1) and f(x2) are compared. If f(x1)<f(x2), then it can be assumed that the breakpoint is upstream from the first endpoint of x2. The location of the breakpoint can be further determined by examining the interval from a to the first endpoint of x2, repeating the steps above. If f(x1)=f(x2)=0, then the breakpoint must be located downstream from the second endpoint of x2. The location of the breakpoint can be further determined by examining the interval from the second endpoint of x2 to b, repeating the steps above. If f(x1)=f(x2)=1, then the breakpoint interval must be located upstream from the first endpoint of x1. The location of the breakpoint can then be even further determined by examining the interval from a to the first endpoint of x1, repeating the steps above. If f(x1)>f(x2), then the breakpoint must be located downstream from the second endpoint of x1 and upstream from the first endpoint of x2. The location of the breakpoint can be further determined by examining the interval from the second endpoint of x1 to the first endpoint of x2, repeating the steps above. Probes are then ideally continually placed and hybridized in the above manner until (b−a)<2ε, but this may be limited by the availability of single copy probe intervals or samples to test. In Example 2, this method was used to determine the location of a breakpoint between base pairs 124,604,661 and 124,630,536 on human chromosome 9 in the ABL1 oncogene region.

Another preferred numerical method of probe selection is referred to as the golden section method. The golden section method is similar to the bisection method in that it uses the same Boolean equation f(x) and has the same solutions. However, the calculation for probes x1 and x2 is slightly different. Instead of bisecting a chromosomal interval from a to b, the interval is instead partitioned using the golden ratio. This partition is calculated in the following manner. First, it is noted that b/(a−b)=a/b. This implies that b*b=a*a−ab. Solving this equation for a yields a=(b square root(5))/2. Accordingly, a/b=0.618 or 1.618.

In accordance with this ratio, probe x1 has a first endpoint that is approximately equal to a+(b−a)*0.382, and probe x2 has a first endpoint that is approximately equal to a+(b−a)*0.618. The probes are then hybridized and the resultant hybridizations are used to solve for the solutions of these equations. The solutions of the functions are then compared with one another. If f(x1)>f(x2), then a new interval is examined between the second endpoint of x1 and b. The methods above are then applied to create two new probes. Mathematically speaking, x1 will equal a in the probe selection equation above, and x2 will equal x1. Accordingly, x2 will equal a+(b−a)*0.618. If f(x1)<f(x2), then a new interval is examined between a and the first endpoint of x2. Mathematically speaking, x2 will equal b in the probe selection equation above, and x1 will equal x2. Accordingly, x1 will equal a+(b−a)*0.382. Probes are then ideally continually placed and hybridized in the above manner until (b−a)<2e, but this may be limited by the availability of probes. In Example 3, this method is used to determine the location of a breakpoint between base pairs 124,632,735 and 124,645,118 on human chromosome 9.

Another preferred numerical method for the selection of probes to determine breakpoints in a chromosome is referred to as the combinatorial method of probe selection. The combinatorial method relies on a function f(x) that has three possible solutions. This is because the combinatorial method of selection relies on a multitude of probes within a chromosomal interval from a to b. These probes are labeled x1 through xₙ, where n is the total number of probes. The combinatorial method relies on the golden section method for the initial selection of probes. Probe x1 has a first endpoint that is located near a+(b−a)*0.382, and probe xₙ has a first endpoint that is located near a+(b−a)*0.618. Probes x2 through x₍ₙ₋₁₎ are located between x1 and xₙ.

After the probes have been hybridized, there are three possible solutions to the function f(x). If all of the probes have moved to a different derivative chromosome, then f(x)=2. In this case, the breakpoint must be located downstream or distal from the first endpoint of x1. Accordingly, a new chromosomal interval is examined in the above manner between a and the first endpoint of x1. If after hybridization, all of the probes remain on the original chromosome, then f(x)=0 and the breakpoint must be located downstream from the second endpoint of xₙ. Accordingly, a new chromosomal interval is examined in the above manner between the second endpoint of xₙ and b. In the case of a split or separated signal—that is, some of the probes remain on the original chromosome and some move to a different chromosome, then f(x)=1. Accordingly, a new chromosomal interval is examined in the above manner between the second endpoint of x1 and the first endpoint of xₙ. Probes can continue to be used until a breakpoint is determined within the desired resolution. In Example 4, this method was used to determine the location of a breakpoint between the IVS1b and IVS3 regions of the ABL1 gene on human chromosome 9.

Yet another preferred numerical method for the selection of probes to determine breakpoints in a chromosome is referred to as the cumulative probability method of probe selection. This method relies on already known breakpoints in a chromosome for a particular disorder in order to find the breakpoint in a particular patient. For example, there have been many breakpoints discovered on human chromosome 9 for patients with chronic myelogenous leukemia (CML). The breakpoints are graphed using a Bayesian function along a chromosomal interval from a to b. Next, a probe x1 is selected that is nearest to the greatest Bayesian maxima in the interval. After hybridization, if probe x1 is determined to have moved to a different chromosome, then the next probe, probe x2, is a probe located nearest the greatest Bayesian maxima between a and the first endpoint of x1. If probe x1 is determined to have remained on the chromosome, then probe x2 is a probe nearest the greatest Bayesian maxima between the second endpoint of x1 and b. Ideally, probes are selected until a breakpoint is determined to at a Bayesian maxima or between two Bayesian maxima. In Example 5, this method is used to determined the location of a breakpoint between nucleotide coordinates 124,623,522 and 124,630,536.

With all of these methods, a plurality of probes can be selected based on the results of the sampling method. In some preferred forms, the probes can be labeled with a plurality of different types of labels, including different colors. Use of multiple label types or colors can facilitate the hybridization and differentiation of several probes within the same experiment. The probe colors can be entirely different or a combination of different colors can be used to provide different color intensities that indicate the delineation of the breakpoint or rearrangement interval. The colors or color intensities can be deconvoluted by optical filters and measured with a device. Preferred devices include spectrometers, photographic apparatuses, laser detectors, or some combination of different devices.

When the selected numerical method determines the coordinates of the next probe to select, it is understood that a probe is not always going to be located precisely at the desired coordinate. In such situations, the closest probe to the desired coordinate is selected as the probe. In the examples herein, this is referred to as “near.”

The present invention finds utility in a variety of hybridization platforms including bead array hybridization, microarray hybridization, fluorescence in-situ hybridization, Southern hybridization, multiplex amplifiable probe hybridization, other probe hybridization techniques, and combinations thereof. Several of these methods permit multiple probes to be analyzed in parallel or rapidly and sequentially while still benefitting from the present invention's teaching of how to select probes in an efficient manner that narrows the breakpoint intervals. FISH techniques are especially suited for examining balanced chromosome rearrangements. Techniques amenable to parallellization will expedite the delineation of breakpoint intervals in patients or specimens with partial aneuploidy.

Using the methods described herein, a number of hybridization probes were identified between the 5′ genome coordinate of the ASS gene and the 3′ coordinate of the ABL1 gene. These identified probes have been verified and include SEQ. ID Nos. 1, 3-13, and 22-56. Those of skill in the art will understand that these probes were selected from a multitude of potential probes that were within this interval of interest.

As used herein, “breakpoint” refers to the precise position within a genome at which two DNA sequences that are not collinear in a reference sequence have been juxtaposed and are adjacent to one another. The term “breakpoint interval” signifies a genomic segment separated by a pair of adjacent sequence-defined probes of known coordinates. A “chromosome rearrangement,” by definition, produces one or more chromosome “breakpoints.” “Chromosome rearrangements” can result in deletions, duplications, amplifications, translocations, inversions, insertions or combinations of these chromosome structures that are not typically observed in normal individuals. Chromosome “abnormalities” are distinguished from chromosomal polymorphisms because abnormalities are considered pathogenic by those of skill in the art. Polymorphisms can be found in normal individuals; however, certain polymorphisms can predispose to presence of abnormalities in offspring of those individuals.

### BRIEF DESCRIPTION OF THE DRAWING FIGURES

The patent or application file contains at least one drawing executed in color. Copies of this patent or patent application publication with color drawing(s) will be provided by the Office upon request and payment of the necessary fee.

FIG. 1 is a schematic diagram that illustrates the expected results of the prior art versus the expected outcomes of the methods of the present invention;

FIG. 2 is a schematic diagram illustrating the structure of the BCR and ABL1 genes in normal patients and the translocations of portions of the genes in patients with leukemia;

FIG. 3 is a schematic diagram of the IVS1B intron of the ABL1 gene illustrating various probes and their coordinates, as well as illustrating part of the experiments of Example 2;

FIG. 4 is a photograph of the hybridization of probes 20 and 21 to chromosome 9 and derivative chromosome 22;

FIG. 5 is a photograph of the hybridization of probe 16 to chromosome 9 and derivative chromosome 9;

FIG. 6 is a photograph of the hybridization of probe 17a to chromosome 9 and derivative chromosome 22;

FIG. 7 is a photograph of the hybridization of probes 25, 27, and 29 to chromosome 9 and derivative chromosome 22;

FIG. 8 is a photograph of the hybridization of probes 25, 27, and 29 to chromosome 9 and derivative chromosome 22 and probes 16 and 18 to chromosome 9 and derivative chromosome 9;

FIG. 9 is a schematic diagram illustrating a color-coded combinatorial labeling method of probe computation;

FIG. 10 is a schematic drawing and table illustrating the effectiveness of various computational methods of probe selection;

FIG. 11 is a graph illustrating the distribution of breakpoints in the IVS1B intron of ABL1 on chromosome 9 in 27 leukemia patients; and

FIG. 12 is a graph illustrating the distribution of known chromosomal breakpoints along the ABL1 gene in chromosome 9.

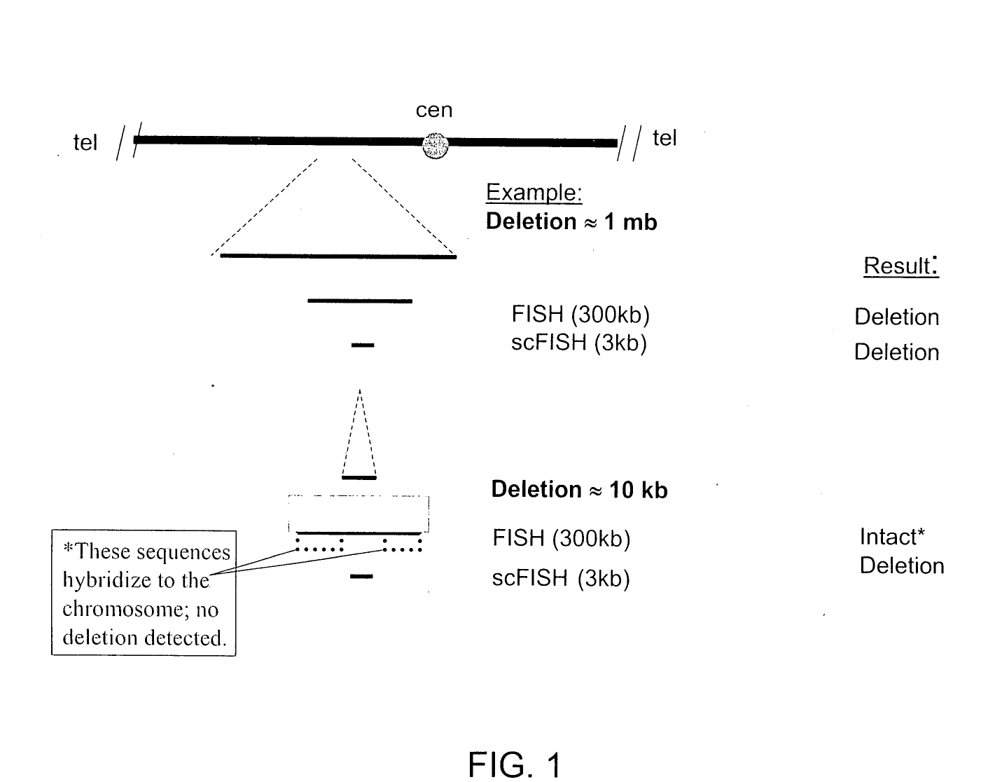

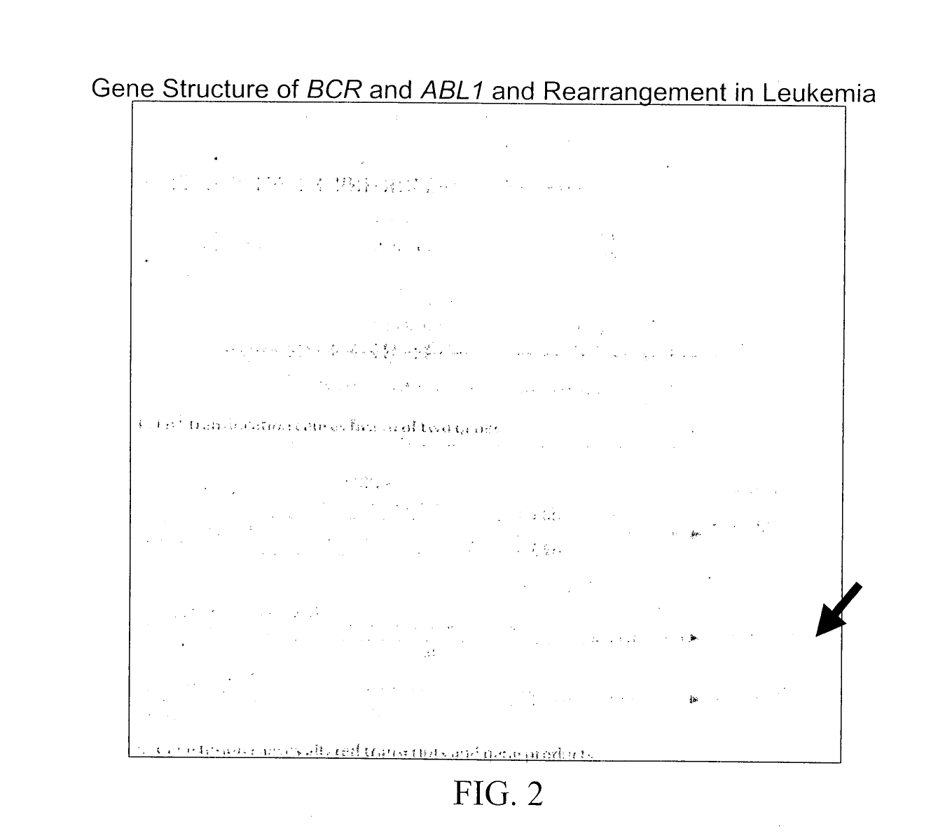

### DETAILED DESCRIPTION OF THE PREFERRED EMBODIMENTS

Since certain changes may be made above method without departing from the scope of the invention, it is intended that all matter contained in the above description and shown in the accompanying drawing shall be interpretive as illustrative and not limiting. It is also understood that the following claims are intended to cover all of the generic and specific features of the invention described herein, and all statements of the scope of the invention which, as a matter of a language might be said to fall between.

### Example 1

This example demonstrates the feasibility of using scFISH probes throughout the human genome and incorporates by reference the teachings and content of Rogan et al., Sequence-Based Design of Single-Copy Genomic DNA Probes for Fluorescence In Situ Hybridization, Genome Research, vol. 11, pp. 1086-1094 (2001). This was accomplished by analyzing the organization of potential single copy probe sequences on chromosomes 21q and 22q. Chromosome 21q contains a lower density of genes than chromosome 22q, and is somewhat more representative of the complete genome. The coordinates of each of the repetitive sequence elements in chromosome 21q and chromosome 22q sequences were located with the computer program CENSOR (as described in Jurka, J., Klonowski, P., Dagman, V., and Pelton, P., “CENSOR—A Program for Identification and Elimination of Repetitive Elements from DNA Sequences”, Computational Chemistry, Vol. 20, pp. 119-122, 1996, which is hereby incorporated by reference). The sequence used for chromosome 21 is available in Hattori et al., “The DNA Sequence of Human Chromosome 21”, Nature, vol. 405, pp. 311-319, 2000, which is hereby incorporated by reference. The sequence used for chromosome 22 is available in Dunham et al., “The DNA Sequence of Human Chromosome 22”, Nature, vol. 402, pp. 489-495, 1999, which is hereby incorporated by reference. The locations and lengths of each intervening interval and the distances separating adjacent intervals were computed using a Perl script (findi.pl). The Perl script was used to deduce and sort the adjacent single-copy intervals by size. These boundaries were deduced by subtracting one nucleotide position from the upstream boundary of a repetitive element and adding one nucleotide position to the downstream boundary of the previous element.

Another Perl script (probsc.pl) was used to determine the lengths of genomic sequences required to find single copy FISH probes exceeding parametrized lengths that were greater than or equal to 2.3 kb. This program is operated by computing the probability of detecting at least one single-copy interval greater than the specified length in every genomic interval on both chromosomes 21q and 22q. For each single-copy window length, a range of genomic windows was tested up to about 220 kb. The chromosomal single-copy interval distributions were then analyzed with SPSS v. 9.0 (SPSS, Chicago, Ill.). This analysis was used to estimate the resolving power of single copy FISH probes for genome-wide studies. The lengths and distances between intervals were plotted on a log scale and their significance was evaluated with the Kolmogarov-Smimov statistic. In this manner, deviations from a normal distribution were obtained.

Chromosome 21 was determined to have fewer single copy intervals than chromosome 22, and the intervals are, on average, shorter. Single copy intervals suitable for use as a FISH probe (for purposes of this example, 2.3 kb or more in length) were determined to be separated, on average, by 29.2 kb on chromosome 21 and by 22.3 kb on chromosome 22. Most of the intervals separated by 1.25 kb to 100 kb on chromosome 22 are normally distributed. However, higher numbers of densely clustered and sparsely populated chromosomal regions were more prevalent than expected (p<0.0001) and occur throughout the genome. Next, the probability of detecting at least one single copy sequence in overlapping, uniform-length genomic intervals on chromosomes 21q and 22q was determined. This knowledge determined the size of single copy segments greater than 2 kb in length were found in most 100 kb genomic regions (99% of the time in chromosome 22; 95% of the time in chromosome 21). Segments greater than 1 kb in length are found at least once per 30 kb (more than 99% of the time). A large proportion of the 218-kb genomic internal did not have any single-copy segments greater than about 4 kb in length on either chromosome 21 (62%) or 22 (24%). Since genes containing single copy segments can be found throughout these chromosomes, and chromosome breakage within or adjacent to genes is the basis for phenotypic differences (pathogenic or non-pathogenic), probe length should be selected that will detect chromosome rearrangements in genes with a high degree of confidence (ie. >95%). Therefore, it was determined that single copy FISH probes for these chromosomes should be 2 kb or less in length to ensure comprehensive coverage (at least once per 100-150 kb) of chromosomes 21 and 22 for detecting rearrangements within or adjacent to gene regions. Accordingly, it will be feasible to develop single copy FISH probes capable of high resolution for molecular cytogenetic analysis of most clinically relevant chromosomal rearrangements.

### Example 2

This example is illustrative of the dichotomous method of bisection of the interval for probe selection using the ABL1 oncogene as an example.

First, bone marrow samples were selected from 71 persons diagnosed with CML and determined to have a translocation between chromosomes 9 and 22 by cytogenetic GTG-banding. In brief, cells from each sample were prepared and chromosomes were digested with trypsin and then stained using a Giemsa staining procedure. After staining, the cells were visually examined with a microscope to determine whether a translocation was present. A proportion of cells from each patient sample were determined to have a 9; 22 chromosome translocation.

In patients with CML, sequences distal to intron 1b (IVS1b) of the ABL1 oncogene on chromosome 9 (which is usually disrupted) and have been translocated to the promoter of the BCR gene on chromosome 22. Additionally, approximately 10% of CML patients also have a disruption upstream from the ABL1 oncogene, resulting in a chromosomal deletion on the derivative chromosome 9. This creates a large deletion on the chromosome—at least 300,000 bp—that must be examined to locate a breakpoint. Given that genomic probes range can be as small as about 50 bp, the cost of determining a breakpoint can be high, especially if a comprehensive set of probes covering the whole interval are prepared and tested. Therefore, a way to optimize the selection of probes was necessary so that the overall number of probes required could be reduced.

A number of probes from within the deletion interval and ABL1 gene (SEQ ID No. 2) were developed to detect small deletions and determine translocation breakpoints. The probes developed focused on known genes with disruptions in SEQ ID No. 2. These probes are provided herein as SEQ ID Nos. 21-36. Of course, not all probes were used for all patient testing as the methods of the present invention, particularly the bisection method, identified the proper probes for each individual patient.

In this example, dichotomous bisection mapping was used to determine a breakpoint in the ABL1 gene. First, two FISH probes were selected as follows. Using a computer, the sequence of the appropriate sequence of chromosome 9 was identified using the Human Genome database (Build 30, National Center for Biotechnology Information, or Genome browser version hg12, June 2002, the teachings and content of which are hereby incorporated by reference). Next, an mRNA sequence for the chromosomal region was identified and was designated SEQ ID. No. 1. The mRNA sequence was compared to the genomic sequence, and the genomic sequence was designated SEQ ID. No. 2. The computer program RepeatMasker was used to determine the locations of repetitive sequences. The Perl script findi.pl was then used to parse the coordinates of the boundaries of the repetitive segments previously identified using RepeatMasker. Each of these programs can be found in U.S. patent application No. 09,854,867, filed May 14, 2001, or Rogan et al., Genome Research 2001 (cited herein), the teachings and content of which are hereby incorporated by reference. Single-copy intervals with identical upstream and downstream coordinates were determined to be adjacent. Using this process, two probes, designated probe 20 and probe 21, were selected as they were located close to the center of the chromosome span in question, which had endpoints of 124,604,542 and 124,725,532. The nucleotide sequence of probe 20 was designated SEQ ID No. 3 and the sequence of probe 21 was designated SEQ ID No. 4.

Probes 20 and 21 are single copy probes that were manufactured in the following manner. DNA fragments corresponding to SEQ ID Nos. 3 and 4 were amplified by long Polymerase Chain Reaction (“PCR”) following the procedure in Cheng, et al., “Effective Amplification of Long Targets from Cloned Inserts and Human Genomic DNA”, Proceedings of the National Academy of Sciences, Vol. 91, pp. 5695-5699, 1994, which is hereby incorporated by reference. The long PCR procedure was performed using LA-Taq (Takara Bio, Inc., Japan) as recommended by the manufacturer (Invitrogen, Carlsbad, Calif.). The resulting amplicons were then purified by low-melt temperature agarose gel electrophoresis. This was followed by chromatography with Micro-spin columns (Millipore, Billerica, Mass.), which removed contaminating extension products containing repetitive sequences. The probes were then labeled by nick translation using modified nucleotide digoxigenin-dUTP, or biotin-dUTP, or both (Roche Molecular Biochemicals, Indianapolis, Ind.).

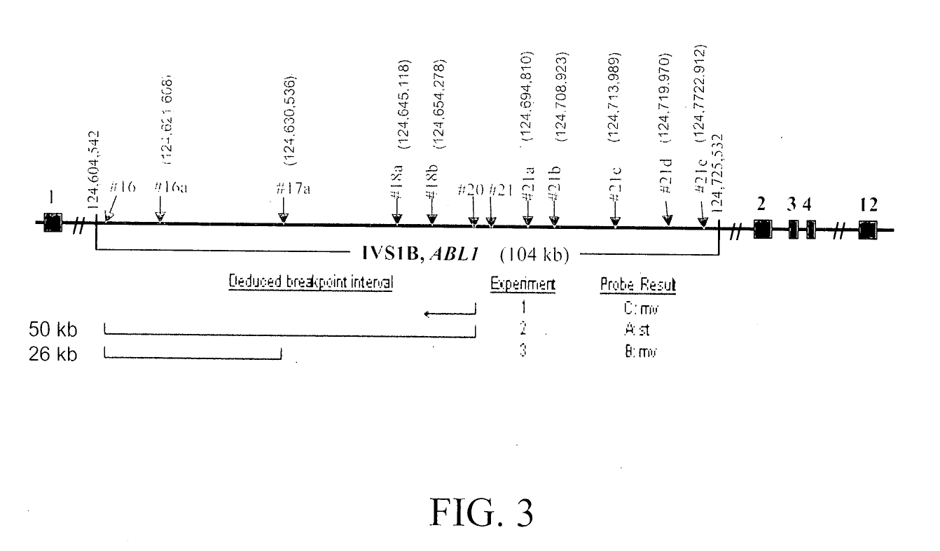

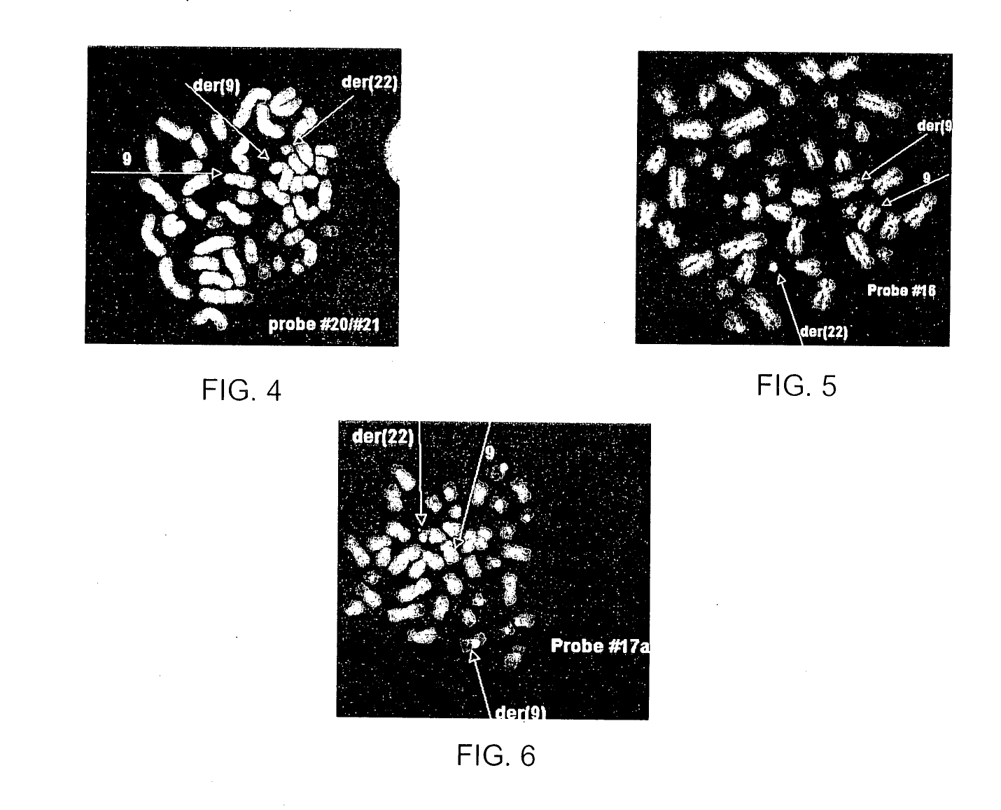

The labeled probes were then denatured and hybridized to fixed chromosomal preparations on microscope slides using the procedure described in Knoll, J. H. M. and Lichter, P., “In Situ Hybridization to Metaphase Chromosomes Interphase Nuclei”, Current Protocols in Human Genetics, Vol. 1, unit 4.3 (eds. N. C. Dracopoli, et al.), John Wiley, New York, 1994, which is hereby incorporated by reference. However, there is one exception from that procedure. In this example, preannealing of the probe(s) with repetitive DNA (such as C₀t1 DNA) was not necessary and was not used. For several other probes containing repetitive sequences (eg. probes 16-1, 16-2, 16-3, 16a, 17a, 18a, 18b, 21-1, 21-2, 21a, 21b, 21c, 21d, and 21e]), the probes were pre-annealed with Cₒt−1 DNA prior to chromosomal hybridization. Probes containing repetitive sequences are denoted in FIG. 3 in red letters. The probes were hybridized individually or in combination to remove nonspecific binding. Posthybridization washes were performed at 42 degrees C. in 50% formamide in 2×SSC (0.3M NaCl, 0.3M 0.03M sodium citrate, pH7.0), followed by an additional wash at 39 degrees C. in 2×SSC and one in 1×SSC (0.15 M NaCl and 15 mM sodium citrate, pH 7.0) at room temperature. The hybridized probes were detected with a fluorochrome tagged antibody to the modified nucleotide and non-specifically bound antibody was removed by additional washing with 1×SSC, 1×SSC with Triton X100, and 1×SSC again, each at room temperature. Chromosome identification was performed by counterstaining the cellular DNA with 4′,6-diamidino-2-phenylindole (DAPI). The hybridized chromosomes were then viewed with an epifluorescence microscope (Olympus, Melville, N.Y.) equipped with a motorized multiexcitation fluorochrome filter wheel. Hybridization patterns were then scored for each probe. The cells were then imaged using a CCD or analog camera (Cohu, San Diego, Calif.) and CytoVision ChromoFluor software (Applied Imaging, San Jose, Calif.). This image is shown here as FIG. 4. Both probes 20 and 21 were determined to have translocated to the derivative chromosome 22, according to the bracketing sampling method, f(x1)=1. Accordingly, it could be deduced that the breakpoint was located upstream or proximal of probe 20 on chromosome 9.

Given this information, another probe was selected whose sequence would hybridize near coordinate 124,604,542, the proximal (or centromeric) endpoint of the chromosomal region known to contain breakpoints within ABL1. The coordinates are derived from Build 30 of the NCBI human genome sequence. This probe was designated probe 16 and its sequence corresponded to SEQ ID No. 5. Probe 16 was manufactured in the same manner as probes 20 and 21, and then hybridized to chromosomes in the patient sample in the same manner as probes 20 and 21. The resulting image is shown here as FIG. 5. Probe 16 was determined to have remained in the derivative chromosome 9, meaning that according to the bracketing sampling method, f(x2)=0. Thus, it could be deduced that the breakpoint in the chromosome was downstream from the location of probe 16 and upstream from the location of probe 20, since f(x1) was >f(x2).

Accordingly, another probe was selected whose sequence would hybridize at about the midpoint between the endpoint of probe 16 and the startpoint of probe 20. This probe was designated 17a and its sequence corresponded to SEQ ID No. 6. Probe 17a was manufactured in the same manner as probes 20 and 21, and then hybridized to chromosomes in the patient sample in the presence of Cot1 DNA, but otherwise in the same manner as probes 20 and 21. The resulting image is shown here as FIG. 6. Probe 17a was determined to have moved to the derivative chromosome 22, meaning that according to the bracketing sampling method, f(x3)=1. Thus, it could be deduced that the breakpoint in the chromosome was downstream from probe 16 and upstream from probe 17a, since f(x3) was >f(x2). Accordingly, the breakpoint in the chromosome lies somewhere in the 26 kb span between base pair 124,604,661 (the end point of probe 16) and 124,630,536 (the start point of probe 17a).

### Example 3

This example illustrates a hypothetical use of the “Golden Section ratio” method of probe selection in a patient with a chromosome 9 translocation at a known breakpoint at 124,643,186.

The section of chromosome 9 used in the previous examples and including the ABL1 gene is selected for breakpoint determination. The total number of base pairs in this span of chromosome 9 is 120,990. When this number is multiplied by 0.618, the result is about 74,772. The first endpoint of this chromosome span is located at base pair 124,604,542, and so a probe should be selected that is near base pair 124,679,314. Accordingly, the first probe selected is probe 21 (as described in Example 2), which has a coordinate of approximately 124,684,469 bp. Probe 21 is then manufactured and hybridized to the chromosomes of the sample as described in Example 2. For those probes containing repetitive sequence, Cₒt−1 DNA is preannealed prior to chromosomal hybridization (see FIG. 3). After hybridization, it is determined that the probe has moved to derivative chromosome 22. Therefore, the breakpoint is located upstream from the region corresponding to probe 21. Thus, according to the bracketing sampling method, f(×1)=0.

Given this information, another probe is selected in the following manner. Again, the total number of base pairs in this interval of chromosome 9 is 120,990. When this number is multiplied by 0.382, the result is 46,218, which corresponds to the distance of the selected probe from one end of the interval. Accordingly, the second probe selected is near base pair 124,650,760 (this is the sum of the endpoint and the computed distance). In accordance with the methods of Examples 1 and 2 above, a probe designated Probe 18a is accordingly selected, which is at base pair 124,645,118 and corresponds to SEQ ID No. 7. This probe is then manufactured and hybridized in accordance with the methods of Example 2. The results indicate that probe 18a moved to derivative chromosome 22, meaning that according to the bracketing sampling method, f(x2)=0. Thus, it could be deduced that the breakpoint in the chromosome was upstream from the location of probes 18a and 21, since f(x2)=f(x1)=0.

Given this information, it can be assumed that the breakpoint interval lies in between base pair 124,604,542 and base pair 124,645,118-a span of 40,576 base pairs. When this number is multiplied by 0.618, the result is about 25,076. Accordingly, the methods of Examples 1 and 2 are then used to select a probe near base pair 124,629,618. Accordingly, probe 17a (as described in Example 2) is selected, since it begins at base pair 124,630,536. When probe 17a is manufactured and hybridized in accordance with the methods of Example 2, it is determined that probe 17a remains on derivative chromosome 9. Thus, according to the bracketing sampling method, f(x3)=1. Accordingly, the breakpoint must be downstream from probe 17a and upstream from probe 18a, since f(x3)>f(x2). Therefore, the breakpoint is within the chromosome span between base pair 124,632,735 (the endpoint of probe 17a) and base pair 124,645,118.

### Example 4

This example illustrates the combinatorial method of probe selection.

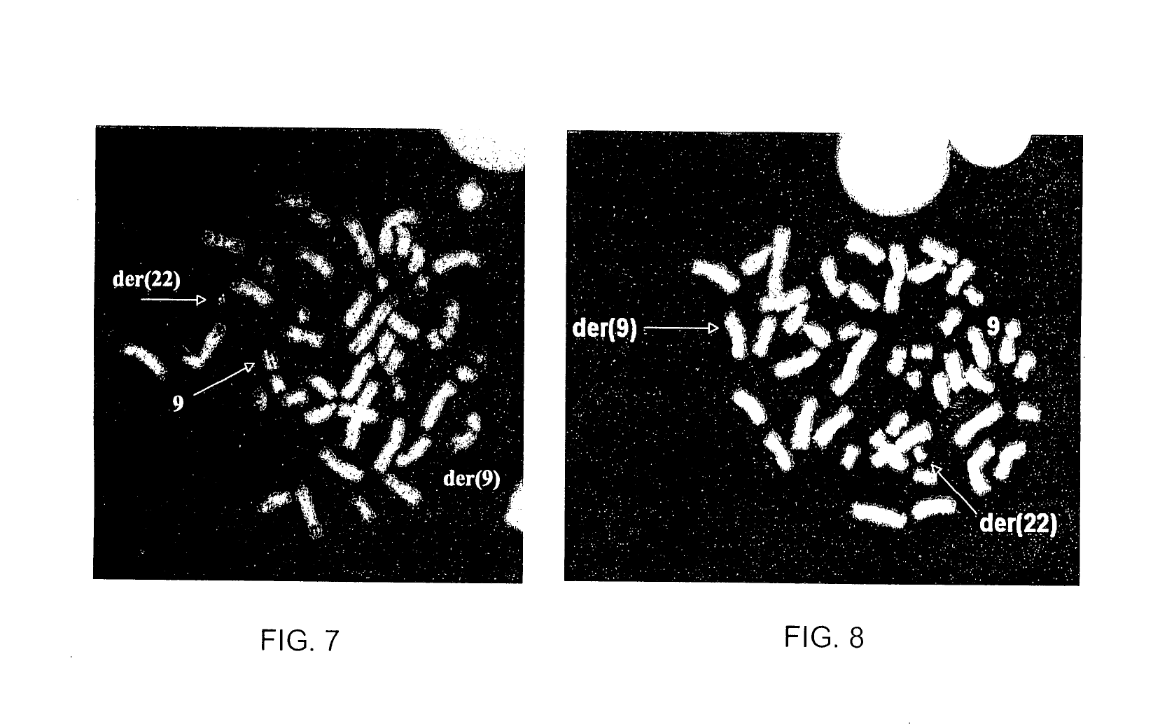

The section of chromosome 9 used in the previous examples and including the ABL1 gene was selected for breakpoint determination. Using the methods described in Example 2, above, three probes were selected and were designated probes 25, 27, and 29. The sequence of probe 25 is in the IVS3 region of the ABL1 gene and corresponded to SEQ ID No. 8. The sequence of probe 27 is in the IVS4-6 region of the ABL1 gene and corresponded to SEQ ID No. 9. The sequence of probe 29 is in the IVS11 region of the ABL1 gene and corresponded to SEQ ID No. 10. Probes 25, 27, and 29 were then manufactured and hybridized to the chromosomes of the sample as described in Example 2. The results of this hybridization may be viewed in FIG. 7. This result indicated that all three of these regions had moved to chromosome 23. Therefore, it was determined that the breakpoint was located upstream of region IVS3 of the ABL1 gene.

In order to further determine the location of the breakpoint, probes 25, 27, and 29 were prepared as noted above and combined with two more probes, probe 16 and probe 18. The sequence of probe 18 was in the IVS1b region of the ABL1 gene and corresponded to SEQ ID No. 11. All 5 probes were then manufactured and hybridized to the chromosomes of the sample as described in Example 2. The results of this hybridization may be viewed in FIG. 8. Again, probes 25, 27, and 29 hybridized to their respective sequences on the translocated chromosome 22 (also known as the “Philadelphia chromosome”), indicating that all three of these probes are downstream of the breakpoint. However, both probes 16 and 18 were hybridized to derivative chromosome 9. To determine whether the chromosome breakage event occurred in the interval separating probes 16 and 18 or the interval separating probes 18 and 25, probe 18 was hybridized individually to the patient's chromosomes. Probe 18 was found to hybridize to the derivative chromosome 9, rather than the translocated chromosome 22 (as well as to the normal copy of chromosome 9 in cells, as expected). Accordingly, it was deduced that the breakpoint was located between probe 18 which occurs within IVS1b and probe 25 which occurs within IVS3 of the ABL1 gene. This interval is bounded by coordinates 124647340 and 124750114 of chromosome 9. It can be appreciated by those of skill in the art that iterative hybridization of additional probes within this region (18a, 18b, 19, 20, 21, 21-1 21-2, 21a, 21b, 21c, 21d, 21e, 22, 23, and 24) either individually or combinatorially, will narrow the breakage interval further so that the breakpoint can be directly identified using, for example, the techniques described in Example 7.

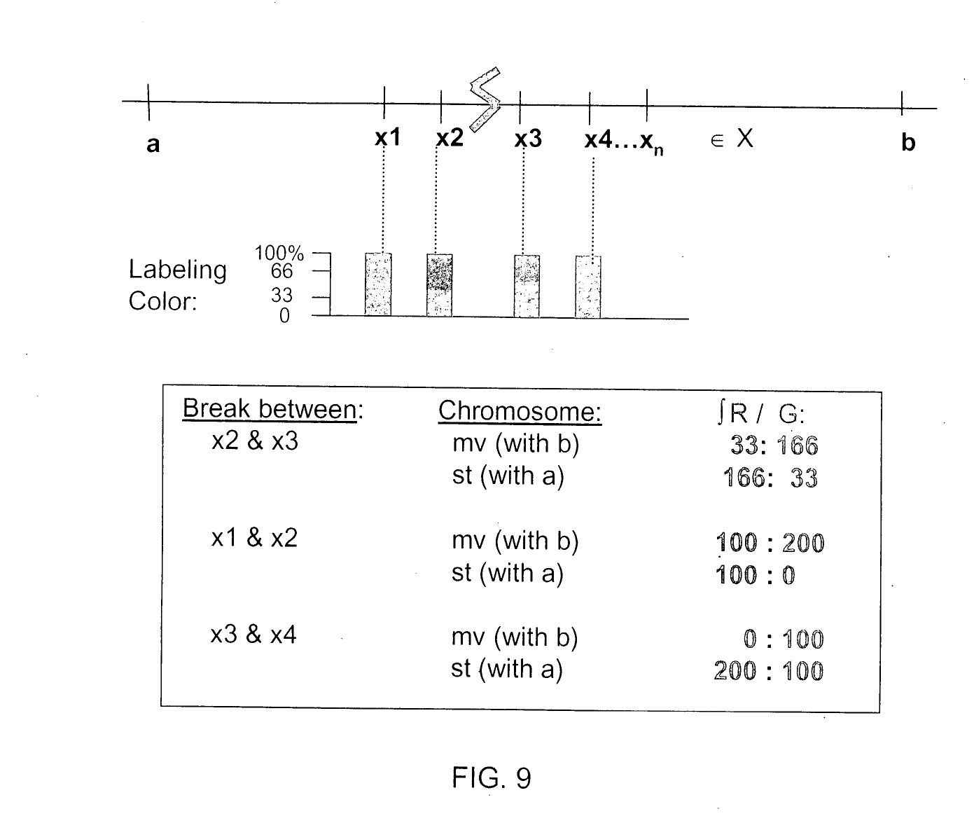

Using this method, there are several means with which the breakpoints can be determined using a probe “cocktail” as described above. One means is to simply use probes that are each tagged with a different color, antigen, or label or mixtures of these colors, antigens or labels. The mixtures or individual tags can be optically separated with appropriate filters as is well known by those of skill in the art. Another means of doing so is to use “color-coded” labeling, as can be seen in FIG. 9. For example, given four probes (x1, x2, x3, and x4), then two colors can be used, such as red and green, to define a translocation breakpoint. The red color would be obtained, for example, by labeling probe DNA by nick translation with biotinylated-dUTP, dGTP, dATP, and dCTP, and detecting the incorporated biotinylated nucleotides with streptavidin conjugated to rhodamine. The green color would be obtained by labeling probe DNA by nick translation with digoxigenin-dUTP, dGTP, dATP, and dCTP and detecting the incorporated digoxigenin modified nucleotides with anti-digoxigenin antibody conjugated with fluorescein. Each x1 probe would be labeled red. The x2 and x3 probes would each consist of mixtures of the biotinylated and digoxigenin labeled DNA. Two-thirds of each x2 probe would be labeled red and the other third of each x2 probe would be labeled green. One-third of each x3 probe would be labeled red and the other two-thirds would be labeled green. Finally, every x4 probe would be labeled green. If all of the probes were simultaneously hybridized to the chromosome, the breakpoint interval can be determined by integrating the color intensity in the resultant chromosomes. For example, if the break is between probes x2 and x3, then derivative chromosome 22 will have an integrated intensity of 33 red:166 green, and derivative chromosome 9 will have an integrated intensity of 166 red: 33 green. The integrated red:green intensities of the resultant chromosome(s) essentially determine which probes have hybridized to a particular chromosome. In the case of a deletion, only one chromosome hybridization will be evident, but the integrated intensity of probe signals of the remaining chromosome can be used to infer the breakpoint interval. The interpretation of these results is distinguished from prior art methods, since each of the probes having defined coordinates and the combination of integrated intensities, therefore delineate a range of coordinates within which the breakpoint resides.

### Example 5

This example uses cumulative Prior probability distribution of known breakpoints to select probes for subsequent patient studies. This is a hypothetical example illustrating the method as it might be applied to a breakpoint in chromosome 9 at approximately 124,625,000.

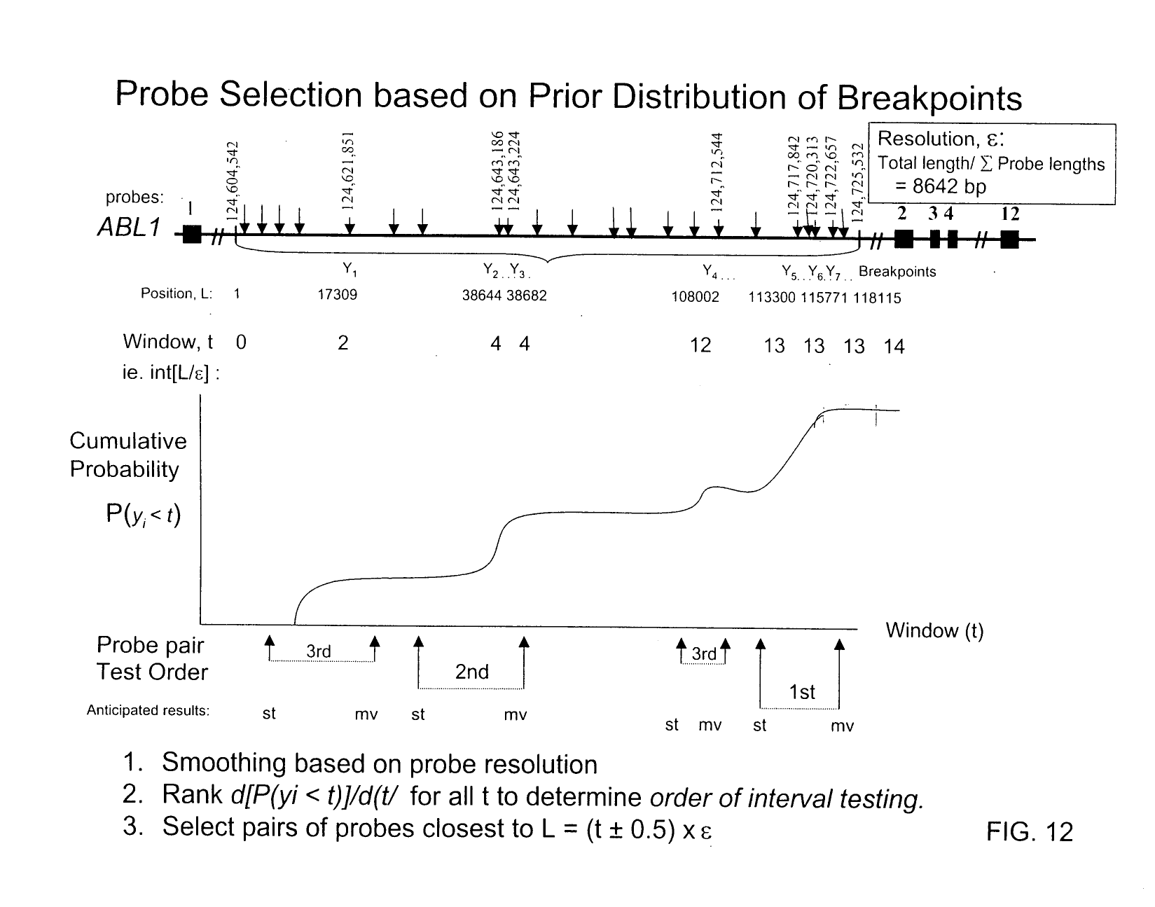

A section of chromosome 9 spanning 120,990 nucleotides from coordinates 124,604,542 through 124,725,532 and including the ABL1 gene is selected for breakpoint determination. Using the methods described in Example 2, a probe designated 21d is selected. Probe 21d corresponds to SEQ ID No. 12 and is located at base pair 124,719,970, which is located near one of the Bayesian maxima as can be seen in FIG. 12. Probe 21d is manufactured and hybridized using the methods of Example 2. After hybridization, it is determined that probe 21d had moved to derivative chromosome 22, meaning that the breakpoint was located upstream from bp 124,719,970.

Accordingly, probe 18a (as described in Example 3) is then selected, since it is located near another Bayesian maxima as can be seen in FIG. 12. Probe 18a is manufactured and hybridized using the methods of Example 2. After hybridization, it is determined that probe 18a moved to derivative chromosome 22, meaning that the breakpoint was located upstream from coordinate 124,645,118.

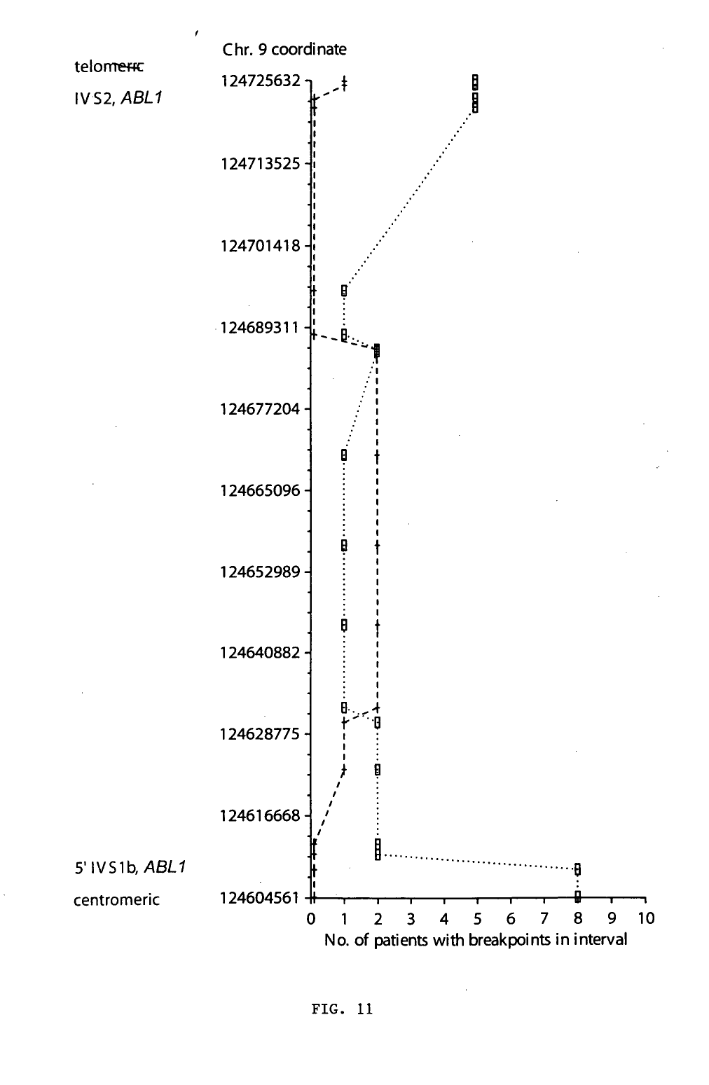

Given this information, another probe, designated 16a, is selected using the methods above. Probe 16a corresponds to SEQ ID. No. 13 and is located near coordinates 124,621,608, which is located near another Bayesian maxima as can be seen in FIG. 11. Probe 16a is manufactured and hybridized using the methods of Example 2. After hybridization, it is determined that probe 16a remained on derivative chromosome 9, meaning that the breakpoint was located downstream from base pair 124,623,522 (the endpoint of probe 16a). Accordingly, probe 17a (as described in Example 2 above) is selected, manufactured, and hybridized using the methods of Example 2. Probe 17a lies between probes 16a and 18a near coordinate 124,630,536. Probe 17a moved to derivative chromosome 22, meaning that the breakpoint was located somewhere in the approximately 7 kb span between coordinates 124,623,522 and 124,630,536.

### Example 6

This example illustrates the relative efficacy of several of the breakpoint determination methods discussed herein. Five methods of breakpoint interval determination were used and compared. The bisection method, the golden section method, the combinatorial method using 5 probes, a combination combinatorial and bisection method, and finally the cumulative distribution method.

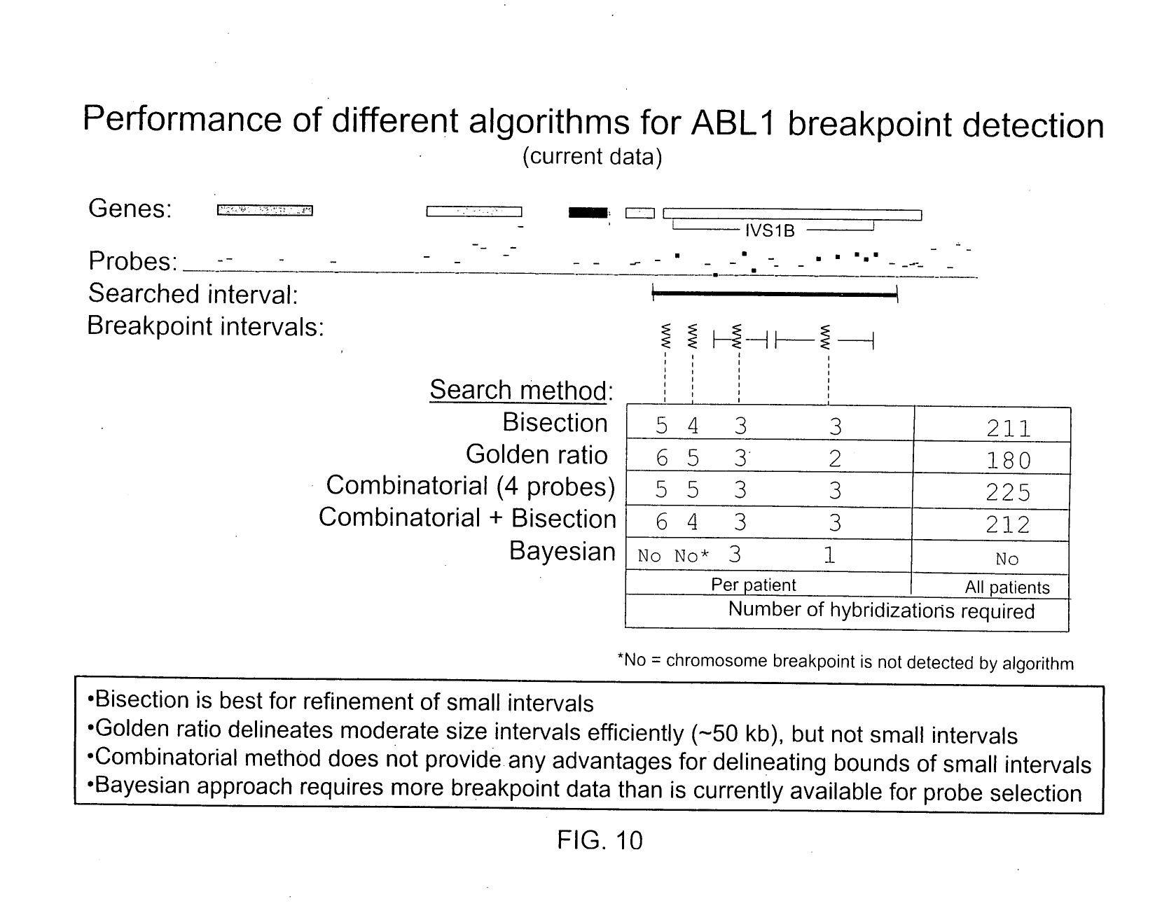

Bone marrow samples were obtained from CML patients and prepared for chromosome/cytogenetic analysis. Chromosome translocations from these patients then had their breakpoint intervals determined using one or more methods described above. A sample from each patient then had its breakpoints determined using the methods described above. The results for these series of breakpoint determinations can be seen as FIG. 10. These results indicate that the bisection method is best for the refinement of small intervals. The golden ratio method is very efficient in determining breakpoints in moderately sized (˜50 kb) intervals, but is not as efficient when determining a breakpoint in smaller intervals. The combinatorial method does not provide any advantages for delineating bounds of small intervals. And with respect to CML, the cumulative prior probability distribution or cumulative distribution approach requires more breakpoint data than is currently available for probe selection.

### Example 7

This example describes one method of identifying a breakpoint directly once a breakage region has been defined using the methods described above.

In order to verify that breakpoint intervals obtained with the instant invention are indeed correct, first, whole genome amplification was carried out using a small aliquot (˜1 μl) of cell pellet from the patient that had been previously fixed in a Camoy's fixative solution for cytogenetic analysis. This amplification followed the procedures of Hosono et al., Unbiased Whole-Genome Amplification Directly From Clinical Samples”, Genome Research, vol. 13(5), pp. 954-964, May, 2003, which is hereby incorporated by reference. The amplified DNA is then extracted twice with phenol and chloroform extractions, and precipitated overnight in ethanol.

The genomic DNA was then digested with restriction endonucleases—ScaI, RsaI, MmeI, and StuI, which were appropriate for the particular breakage regions. The enzymes were identified by simulated restriction digests of the sequence of the targeted breakpoint region. These enzymes cleave DNA in a manner that results in specific cohesive ends, determined from the reference genome sequence, that are amenable to ligation by primers for vectorette PCR. The target was deduced using the known sequence of the breakpoint region that had been delineated by FISH with adjacent probes using the bisection method of Example 2.

The strategy for verifying the breakpoint takes advantage of the restriction site distribution in the translocated chromosome and the absence of restriction sites in the breakage interval of the original chromosome, as well as the known sequence of the targeted region. Using this principle, vectorette “bubble” primers were annealed to the digested genome to produce a vectorette cassette in accordance with the method of Gorenen et al., “Isolation of Cosmids Corresponding to the Chromosome Breakpoints of a De Novo Autosomal Translocation, t(6; 19)(p21; q13.1) in a Patient with Multicystic Renal Dysplasia”, Cytogenet Cell Genetics, vol. 75(4), pp. 210-215, 1996, which is hereby incorporated by reference. The vectorette cassette was then ligated to the patient's digested DNA using “ligation cycling.” Using this technique, the ligated vectorette cassette was incubated for 20 degrees C. for one hour, and then incubated at 37 degrees C. for 30 minutes. This technique assisted the increase of the proportion of ligated products with vectorette units. This is because the presence of the restriction enzyme will result in the cutting of compatible ends and the restriction site is not reconstituted by ligation of the vectorette to the patient's digested DNA.

Next, one cycle of PCR amplification was carried out for the target sequence using a forward primer within the breakage interval. Then, the reverse vectorette-specific primer was added to the PCR reaction. The reverse primer is the reverse complement of the antisense strand of the vectorette in the “bubble” region. This primer will only anneal to the genomic target if the first PCR cycle as described above produced a binding site complementary to the sequence of the reverse primer. Nested PCR was then performed using a second forward primer situated immediately downstream of the first forward primer in the target region.

Then, gel electrophoresis of amplification products was carried out to determine whether both the normal and abnormal chromosomal DNA products had been amplified. The presence of two fragments on the gel, which corresponded to amplicons of different sizes, were determined to be likely to indicate that one is derived from the normal chromosome and one from the abnormal chromosome. The size of the normal chromosome fragment can usually be inferred from the computational restriction analysis of the reference genome sequence. However, it is possible for polymorphisms or other differences between the patient's DNA and the reference sequence to be present. Accordingly, this necessitated that DNA be extracted from both bands. After extraction, the DNA samples were purified with a PCR Clean-Up Kit (Qiagen, Valencia, Calif.). The DNA concentration was then quantitated using a UV Spectrophotometer and compared with the quantitation results from gel electrophoresis for accuracy. The fragments were then sequenced commercially (MWG Biotech, High Point, N.C.) using both vectorette-specific and nested PCR primers.

To confirm breakpoints on chromosome 9 for patients with CML, the electropherograms of the sample DNA and the sequences obtained were analyzed for homology to ABL and BCR genomic sequences. This comparison was made using the BLAT tool from the UCSC Genome Browser (found at genome.usc.edu) and the BLAST server at the NCBI (found at ncbi.nlm.nih.gov). One of skill in the art would appreciate that if the quality of the sequence was determined to be poor, then any sequence obtained would not exhibit similarity to the expected target. If that were the case, then it would be necessary to amplify the genomic library again by PCR using different nested PCR products.

This approach was used to confirm the localization of the breakpoint by the iterative application of the numerical bisection methods of the present invention in 10 patients. Genomic DNA was extracted from lymphoblastoid cell pellets from patients identified by the following codes: 52, 61, 118, 87, 38, 77, 133, 177, 43, and 45. All of the chromosome 9 breakage intervals in these patients have been previously narrowed by iterative application of the instant invention and the chromosome 9 breakpoints have been verified as occurring within these intervals. For brevity, the following describes the procedure used to ascertain the sequences at or near the breakpoints in patients 38 and 77. In order to increase the quantity of DNA available for characterizing the breakpoints in these patients, the 2 ul of DNA from each cell pellet was used in whole genome amplification as described above. This produced approximately 30 ug of replicate genomic DNA. Prior art studies of the this method have verified that the fidelity of replication is extremely high, so that the synthetic copies of these patient's genome are virtually identical to those found in the original samples. The synthetic DNA was extracted twice with phenol/chloroform to remove enzymatic contaminants and buffers and precipitated with ethanol prior to the next step. Genomic DNA was resuspended and incubated with restriction enzymes that were selected because the presumed breakage interval is deficient in these recognition sites. The mapped breakage interval in patient 38 on chromosome 9 comprised coordinates 124685740 through 124686078. Genomic DNA from this patient was digested with Rsa I and ScaI, neither of which cleave within this interval. The mapped breakage interval in patient 77 comprised coordinates 124604661 through 124608696 and this genomic DNA was cleaved with Mme I, Rsa I and Sca I. The bubble vectorette primer (which is complementary to BUB1 sequence) was ligated to these digested DNAs (reference), creating a vectorette library.

In the first round of PCR, the BUB-1 sequence (SEQ ID No. 14) (see, Zhang J G, et al., Characterization of Genomic BCR-ABL Breakpoints in Chronic Myeloid Leukemia by PCR, 90 Br. J. Hematology, 138-146 (1995), the teachings and content of which are hereby incorporated by reference), and the “16.16-1 break” primers (SEQ ID No. 15) were used to amplify the vectorette library in patient 77. The same BUB1 sequence (SEQ ID No. 14) and the 21DownF primers (SEQ ID No. 16) were used in the first round of amplification in patient 38. The second round of amplification in patient 77 used the BUB-2 and 144-2618 primers, whose sequences fall within the interval spanned by the BUB-1 (SEQ ID No. 14) and “16.16-1 break” primers (SEQ ID No. 15). In patient 77, as expected, two products were synthesized corresponding to the normal chromosome 9 sequence (585 bp) and the derivative 9 sequence (˜340 bp) (based on gel electrophoresis standards). The second round of amplification in patient 38 used the BUB-2 (SEQ ID No. 17) and 21DownFNest primers (SEQ ID No. 18). In patient 38, two products were also synthesized, namely ˜890 bp for the normal chromosome and ˜500 bp (based upon gel electrophoresis standards) for the abnormal derivative chromosome. The fragment corresponding to the abnormal band was extracted and reamplified with the nested primers for each patient. The reamplified products were recovered from agarose gels, purified using a commercially available PCR cleanup kit (Quiagen), quantitated by TV spectrophotometry, and submitted for dideoxy sequence analysis by a commercial laboratory (MWG). The nested primers (BUB-2 (SEQ ID No. 17) and either 144-2618 (SEQ ID No. 19) [for patient 77] or 21DownFNest (SEQ ID No. 18) [for patient 38] were used in the sequencing reactions.

After comparing the patient's DNA with the known sequences, a 161 nucleotide amplicon found in chromosome 9 (SEQ ID No. 20) from the patient was found to nearly precisely match (99.4% match) a unique genomic location on chromosome 22 in the BCR gene (positions 26,217,115 to 26,217,268) over 154 bp in patient 38. This process was also used on another patient 77's sample. In that case, a 357 nucleotide sequence (SEQ ID No. 21) obtained by vectorette PCR was found to nearly precisely match (99.7%) a unique genomic location on chromosome 9 in the ABL1 gene (positions 128,870,044 to 128,870,386) over 344 nucleotides. Accordingly, these provided evidence that the sequences came from the translocation partner chromosome 9. Thus, the sequences mapped derive from the expected genomic interval. Therefore, the present invention provides a reliable means of determining breakpoints in a translocated chromosome.

### Example 8

This example illustrates one of the differences between the present invention and the prior art. Breakpoint intervals for 27 patients with CML were determined using the methods of Example 2 using a span on chromosome 9 between coordinates 124,604,561 and 124,725,632 bp. The distribution of these breakpoint intervals can be seen in FIG. 11. As can be seen from this figure, breakpoints are not uniformly distributed along this approximately 120 kb sequence. Furthermore, this figure indicates that the distribution of previously known breakpoints obtained using conventional molecular genetic approaches (which are considerably fewer in number than that obtained with the present invention) is nearly uniform across IVS1b of ABL1 and flanking exonic regions. This contrasts with the non-uniform distribution of breakpoint intervals determined using the present invention, indicating a bias toward breakage at each end of IVS1b. This distribution of breakpoints could not have been anticipated or even determined using commercially available FISH probes, as many commercial recombinant FISH probes for this genomic sequence substantially exceed the length of the breakpoint region, which is the approximate length of this intron. Thus, these breakpoint distributions could not have been discerned using the methods of the prior art.

The 27 breakpoint intervals shown in FIG. 11 were found using methods of the present invention in a period of about 18 months. In contrast, prior art methods identified just 9 breakpoints over a 40 year period. It is clear that the present invention provides distinct efficiency advantages over the prior art methods.

We claim:

1. A method of selecting a genomic hybridization probe comprising the steps of:
(a) selecting a genomic interval of interest;
(b) identifying a plurality of potential hybridization probes of known coordinates in said interval;
(c) applying a numerical method to sample said plurality of probes based on their genome coordinates; and
(d) selecting a probe or probes based on the results of said numerical method.

2. The method of claim 1, said genomic hybridization probe being a single copy genomic probe.

3. The method of claim 1, said method further comprising the step of hybridizing said selected probe with said selected interval.

4. The method of claim 3, further comprising the step of hybridizing blocking DNA to disable hybridization of repetitive sequences with said probe of interest.

5. The method of claim 1, said sampling method being selected from the group consisting of the dichotomous methods including bisection and golden section methods, and combinatorial bracketing, cumulative prior probability distributions of probes, and combinations thereof.

6. The method of claim 1, said probe being labeled.

7. The method of claim 1, including the step of selecting a plurality of probes based on the results of said numerical method.

8. The method of claim 7, said plurality of selected probes being labeled with a plurality of different labels.

9. The method of claim 8, said plurality of different labels producing different colors or color combinations.

10. The method of claim 9, said colors or color intensities being deconvoluted by optical filters and measured with a device selected from the group consisting of spectrometers, photographic apparatuses, laser detectors, and combinations thereof.

11. The method of claim 1, said interval of interest being associated with a chromosomal breakpoint.

12. The method of claim 1, said method further comprising the step of repeating steps (a)-(d).

13. The method of claim 1, said probe being used in a hybridization platform selected from the group consisting of bead array suspension hybridization, microarray hybridization, fluorescence in-situ hybridization, Southern hybridization, multiplex amplifiable probe hybridization, and combinations thereof.

14. The method of claim 1, said genomic hybridization probe being composed of both single copy and repetitive sequences.

15. The method of claim 1, wherein said genomic interval of interest spans from the 5′ genomic coordinate of the ASS gene to the 3′ genomic coordinate of the ABL1 gene.

16. The method of claim 1, said genomic hybridization probe being selected from a group consisting of SEQ. ID Nos. 1, 3-13, and 22-56.

17. The method of claim 1, further comprising the step of detecting one or more chromosome rearrangements to delineate one or more chromosome breakpoint intervals.

18. The method of claim 1, said genomic hybridization probe being capable of hybridizing with plant DNA.

19. The method of claim 1, said genomic hybridization probe being capable of hybridizing with animal DNA.

20. The method of claim 19, said animal DNA including human DNA.

21. A genomic hybridization probe selected by the method of claim 1.

22. The genomic hybridization probe of claim 21, said probe being selected from a group consisting of SEQ. ID Nos. 1, 3-13, and 22-56.
